(19) United States
(12) Patent Application Publication
(10) Pub. No.: US 2007/0055206 A1
(43) Pub. Date: Mar. 8, 2007

(54) Methods and devices for deployment of tissue anchors

(75) Inventors: John To, Newark, CA (US); Mariel Fabro, San Jose, CA (US); Karl S. Im, San Jose, CA (US)

Correspondence Address: MORRISON & FOERSTER LLP, 755 PAGE MILL RD, PALO ALTO, CA, 94304-1018, US

(73) Assignee: Guided Delivery Systems, Inc., Santa Clara, CA (US)

(21) Appl. No.: 11/201,949

(22) Filed: Aug. 10, 2005

(51) Int. Cl. A61M 5/00

(52) U.S. Cl. 604/191

(57) ABSTRACT

Described here are devices, methods, and kits for deployment of tissue anchors. In some variations, the devices described here comprise a shaft defining a lumen for housing at least one anchor therein (the anchor having an eyelet) and a mechanism for deploying the anchor distally from the lumen, wherein the inner diameter of the lumen is the same size or smaller than the diameter of the eyelet of the anchor to be disposed therein when the anchor is in an expanded configuration. In some variations, the methods comprise loading an anchor within a lumen of a shaft (where the anchor comprises an eyelet and the shaft has a slot therethrough), passing a linking member through the slot and through the eyelet of the anchor, and deploying the anchor. Other methods comprise loading an anchor within a lumen of a shaft, and deploying the anchor distally from the lumen.

### FIELD

The devices, kits, and methods described here are in the field of surgery. More specifically, the devices, kits, and methods described here are in the field of tissue anchor deployment.

### BACKGROUND

There are many types of tissue anchors currently known or under development. These anchors range in design from simple staples or T-bars, to complex designs having hooks or barbs. These anchors may be used to modify tissue (e.g., by changing the configuration of the tissue), to fasten one piece of tissue to another, to fasten tissue to material, and the like.

Accordingly, it would be desirable to have devices for delivering tissue anchors that could be used in a variety of procedures, both surgical and percutaneous. It would also be desirable to have devices that are easy to use. Similarly, it would be desirable to have devices that are capable of accessing hard to reach tissues.

### BRIEF SUMMARY

Described here are devices, methods, and kits for deployment of tissue anchors. These devices, methods, and kits may be used in a variety of procedures, both percutaneous and surgical. In some variations, the devices described here comprise a shaft defining a lumen for housing at least one anchor therein (the anchor having an eyelet) and a mechanism for deploying the anchor distally from the lumen, where the inner diameter of the lumen is the same size or smaller than the diameter of the eyelet of the anchor to be disposed therein when the anchor is in an expanded configuration.

In some variations, the distal tips of the described devices have a slot therethrough. In other variations, the devices further comprise a distal tip shoulder. The distal tip shoulder may also have a slot therethrough, and in some variations, the slot of the distal tip and the slot of the distal tip shoulder align axially so that an anchor may be loaded and disposed therein.

The distal tip shoulder may comprise a region of the shaft (i.e., be integral with the shaft), or the distal tip shoulder may be separate and distinct from the shaft. Similarly, the distal tip may be separate and distinct from either the distal tip shoulder or the shaft, or both. The distal tip may also be integral with the shaft or distal tip shoulder, or both.

In some variations, the distal tip of the shaft extends beyond the distal tip shoulder, and in these variations, the portion of the shaft extending beyond the distal tip shoulder may have a smaller outer diameter than the portion of the shaft not extending beyond the distal tip shoulder. The distal tip of the shaft may extend beyond the distal tip shoulder by any desirable distance, and this spacing generally depends upon the anatomy of the structure receiving the anchor. For example, in variations where the anchors are used to repair the mitral valve, the distal tip may extend beyond the distal tip shoulder by about 1 to about 3 mm.

The devices described here may have shafts made of a flexible material, which would be particularly useful during percutaneous procedures. In these variations, the devices usually have a lower profile, consistent with their manipulation through the vasculature. Any suitable flexible material may be used. For example, the material may be selected from the group consisting of nylon, nylon blends, nickel titanium alloys, polyethlyene, polyetheretherketone, polyether block amides, polytetrafluoroethylene, fluorinated ethylene propylene copolymer, stainless steels, polymer blends with or without a supporting metal braid or coil, combinations thereof, and mixtures of one or more of such materials.

The devices may also have shafts made of a rigid material, which would be particularly useful during open or surgical procedures, where access to the target site is achieved by incision. The rigid material may be selected from the group consisting of stainless steel, nickel titanium alloys, carbon-filled nylon, carbon-filled polyetheretherketone, polypropylene, high density polyethylene, combinations thereof, and mixtures of one or more of such materials.

The devices described here may also have a sleeve surrounding (or releaseably coupled to) at least a portion of the shaft. The sleeve may be useful in promoting tissue ingrowth near the target site as well as in providing additional structure and integrity to the area. The sleeve may be made from a material selected from the group consisting of a braided polyester, a woven polyester, polyurethane, polypropylene, a nickel titanium alloy, stainless steel, expanded polytetrafluoroethylene, and mixtures thereof.

The devices described here may also have at least one preformed curve in the shaft. For example, the devices may have a curve near their distal tip. This may help in accessing otherwise difficult to reach areas. The curve may have any suitable angle. For example, the curve may have an angle ranging from about 15 to about 90 degrees.

The device may also comprise at least two shafts, so that multiple anchors may be deployed simultaneously. Similarly, a single shaft may be configured to receive at least two anchors therein, for deploying multiple anchors serially or sequentially. In a like manner, the device may also comprise an additional shaft, or an additional lumen within a single shaft, that is configured to inflate a balloon. This for example, may aid in the deployment of the tissue anchors. The balloon may be made from a material selected from the group consisting of nylon, polyethylene, polyurethane, combinations thereof, and mixtures of one or more of such materials. The devices may have any suitable mechanism of deploying the anchors from the distal end of the lumen shaft. For example, the mechanism may be a hydraulic mechanism, or a pressurized air mechanism. In some variations, the mechanism is a plunger slidably disposed within at least a portion of the lumen, and the device further comprises an actuator for actuating the plunger.

Also described here are kits for the deployment of tissue anchors. In general, the kits comprise a device comprising a shaft configured to deploy anchors distally therefrom, and a linking material configured to couple the anchors together. Any of the devices described here may be used in the kit. Similarly, the linking material may be any material suitable for coupling anchors together. For example, the linking material may be a suture, a thread, a string, a piece of fabric, or the like. In some variations, the linking material is a suture. The kits may also comprise instructions on using the kit. The components of the kit will often be packaged together.

Methods for deploying a tissue anchor are also described. In some variations, the methods comprise loading an anchor within a lumen of a shaft (where the anchor comprises an eyelet and the shaft has a slot therethrough), passing a linking member through the slot and through the eyelet of the anchor, and deploying the anchor. In these variations, the methods may further comprise placing a sleeve over the linking member (e.g., by sliding a sleeve over the linking member or the like). The methods described here also contemplate retrieving the anchor in the event of misplacement.

Other methods described here comprise loading an anchor within a lumen of a shaft (where the anchor comprises an eyelet), and deploying the anchor distally from the lumen, wherein the inner diameter of the lumen is the same size or smaller than the diameter of the eyelet of the anchor to be disposed therein when the anchor is in an expanded configuration. In some variations, when the anchor is deployed distally from the lumen, the anchor is deployed distally from the lumen into a sleeve and the tissue, where the sleeve is releasably coupled to the shaft and where the anchor couples the sleeve to the tissue. The sleeve may also comprise a cinching member, and the method may further comprise applying tension to the cinching member. In yet other methods, the tissue is deployed by loading an anchor within a lumen of a shaft and deploying the anchor distally from the lumen, where deploying the anchor distally from the lumen further comprises deploying the anchor distally from the lumen into a sleeve and the tissue. In these variations, the sleeve is releasably coupled to the shaft and the anchor couples the sleeve to the tissue.

### BRIEF DESCRIPTION OF THE DRAWINGS

FIG. 1 provides an exemplary illustration of a surgical anchor deployment device, where the shaft is rigid.

FIG. 2 provides an exemplary illustration of a percutaneous anchor deployment device, where the shaft is flexible.

FIGS. 3A-3C depict variations on the distal end of one illustrative anchor deployment device as described herein.

FIGS. 4A and 4B show one variation of an anchor deployment device further comprising a sleeve.

FIG. 5 shows a variation of an anchor deployment device where the device comprises at least two shafts.

FIG. 6 provides an exemplary illustration of anchors that have been dipped, coated, or partially potted in a polymer.

FIG. 7 provides an exemplary illustration of one method of deploying the anchors distally from a device, when the device further comprises a sleeve.

FIGS. 8A and 8B depict exemplary illustrations of retrieval aspects of the deployment device.

### DETAILED DESCRIPTION

Described here are devices, kits, and methods for deploying tissue anchors. In general, the devices, kits, and methods may be used with a variety of different anchors and in a variety of different procedures. For example, the devices may be used with anchors of any desirable size, the size of the anchor being largely dependent upon the procedure to be carried out. The devices may be used in percutaneous procedures where access to the anchor deployment site is achieved intravascularly. Similarly, the devices may be used in open surgical procedures where access to the anchor deployment site is achieved via incision.

The devices, kits, and methods described here may be used in the fields of general surgery, cardiology, urology, neurosurgery, gastroenterology, and the like. Exemplary procedures include repair of heart valves (e.g., mitral, tricuspid, aortic), repair or reduction of sphincters, closure of wounds, and in reducing the circumference of the gastroesophogeal junction.

Devices

The devices described here are for the deployment of tissue anchors. In general, the devices may be made of a flexible material having a low profile for percutaneous procedures, or may be made of a rigid material having a larger profile for use in surgical procedures. The devices are configured to deploy tissue anchors, and as such, may be useful in any variety of procedures, including those procedures mentioned above. The devices may be especially useful in deploying anchors in areas of the body that are somewhat difficult to access.

Some of the described devices comprise a shaft having a lumen for housing at least one anchor therein, and a mechanism for deploying the anchor distally from the lumen, where the inner diameter of the lumen is the same size or smaller than the diameter of the eyelet of the anchor to be disposed therein when the anchor is in an expanded configuration. The corresponding anchor has an eyelet and an expanded configuration and a collapsed configuration. For example, when the anchor is in a collapsed configuration, it has a smaller profile, which enables it to be housed within the lumen of the device shaft. When the anchor is deployed from the distal end of the lumen, it assumes an expanded configuration as it expands and secures into tissue. In some variations, having the inner diameter of the lumen smaller than the diameter of the eyelet of the anchor to be disposed therein is useful because it allows the legs of the anchor to assume a more linear shape when the anchor initially exits the lumen. The legs of the anchor then assume a more curved configuration as full expansion occurs.

The anchors to be housed within the lumen of the shaft may be made of any material allowing for a collapsed and expanded configuration, and may be of any suitable size. The size of the anchor selected will depend largely upon the end use of the anchor. For example, anchors to be used in the repair of cardiac valves will be much smaller in dimension than those anchors used to repair large wounds or to reduce the circumference of a large hollow body organ. The anchors may be made, for example, from shape memory or superelastic materials, biodegradable polymers, metals, alloys, or mixtures thereof. Exemplary anchors for use with the devices, kits, and methods described here are those taught in commonly owned co-pending patent application, U.S. Ser. No. 11/______,______ entitled, “Devices and Methods for Anchoring Tissue” filed on Aug. ______, 2005, which is hereby incorporated by reference in its entirety.

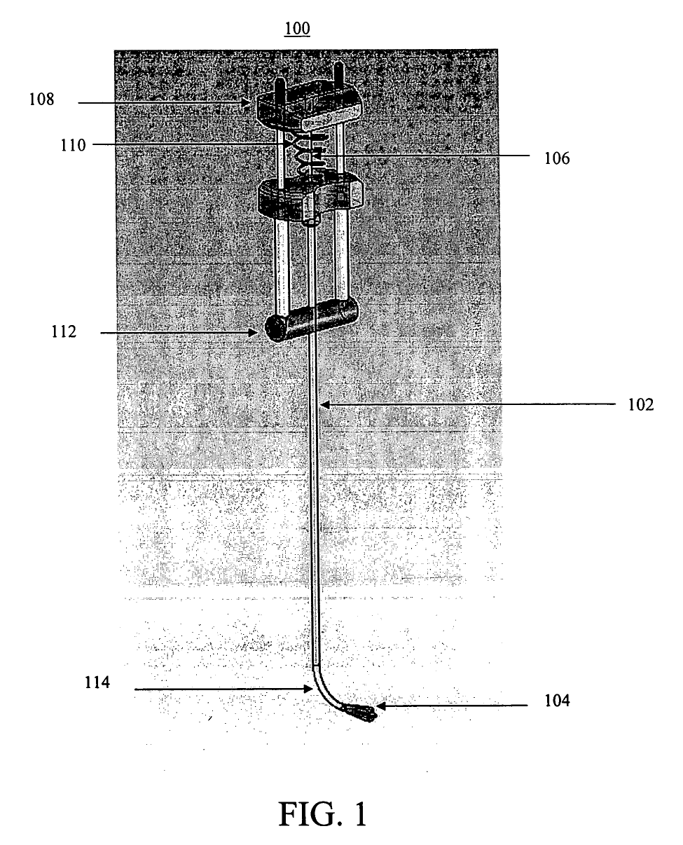

FIG. 1 provides an illustrative depiction of a surgical device (100) for the deployment of tissue anchors. As shown there, device (100) comprises a shaft (102) defining a lumen (not shown), and a mechanism for deploying the anchor distally from the lumen. The lumen may have any suitable cross-section. For example, the lumen may have a circular cross-section, an elliptical cross-section, a rectangular cross-section, or any other geometrically desirable cross-section. When referring to the inner diameter of the lumen in those instances in which a non-circular cross-section is used (e.g., when an elliptical cross-section is used), it is meant the length of the major axis of the lumen.

The mechanism for deploying the anchor distally from the lumen in the variation depicted in FIG. 1 is a plunger (106) slidably disposed within at least a portion of the lumen. In this variation, the device also comprises an actuator for actuating the plunger, shown here as a slidable handle (108) attached to spring (110). Plunger (106), slidable handle (108), and spring (110) may be formed as a single integral unit, or may be formed of several parts and interconnected.

It should be understood that while the slidable handle (108) is shown having a particular geometry or shape in FIG. 1, any suitable shape may be used. It may be desirable, however, to have slidable handle (108) be of an ergonomic shape, so that it is comfortable for operation by depression with a user's thumb, for example. In operation, the user would depress slidable handle (108) causing compression of spring (110) and slidable movement of plunger (106) axially within the lumen of shaft (102).

While the mechanism for deploying an anchor distally from the shaft lumen depicted in FIG. 1 is a plunger slidably disposed within the lumen, any suitable mechanism for deploying an anchor distally from the shaft lumen may be used with the devices, kits, and methods described here. In a like manner, it is not necessary to have a spring when the mechanism is a plunger. The mechanism may be a hydraulic mechanism, a pressurized air mechanism, or any other mechanism capable of providing an axial force on the anchor, sufficient to deploy it distally from the lumen. Similarly, all or a portion of the anchor may be made from, or coated or embedded with, a magnetic material, and a corresponding magnet (e.g., a magnet on the tip of a catheter) may be placed distally of the anchor to withdraw the anchor from the lumen. Additionally, any suitable component may be used as part of such a mechanism (e.g., pistons, plungers, cables, pumps, etc.), and the mechanism for deploying the anchor may be made from any suitable material, such as stainless steel, nickel titanium alloy, nylon, polyetheretherketone, polyether block amides, a TEFLON® polymer, mixtures thereof, and the like. In one variation, the mechanism for deploying the anchor is a push cable made of stainless steel, coated at least in part with a TEFLON® polymer, which may help to reduce friction when sliding within the shaft.

The mechanism for deploying anchors may also be reinforced along those sections that do not traverse curves within the device shaft (e.g., in the case of a push cable, the cable may be reinforced along its straight length). The mechanism may be reinforced with any sutiable material, e.g., a metal or polymer tubing may be used. Similarly, the distal end of the mechanism may be reinforced with an element that helps impart the axially force transmitted from the actuation onto the collapsed anchor.

Also shown in FIG. 1 is distal tip (104), from which the anchor will be deployed, and distal curve (114). While the variation shown in FIG. 1 has a distal curve, the devices described here need not have one. However, it may be useful for the device to have at least one preformed curve. For example, it may be desirable to have a preformed curve near the distal end of the shaft to help access hard to reach areas. The curve may be of any suitable or desirable angle. For example, it may be from about 15 degrees to about 90 degrees, from about 45 degrees to about 80 degrees, or from about 50 degrees to about 70 degrees. Of course, the device may also comprise more than one curve.

In the variation shown in FIG. 1, the shaft (102) is rigid or made from a rigid material. Such a device may be particularly useful in surgical applications where an incision is used to access the site for anchor deployment. Any suitable rigid material may be used. For example, the material may be made from stainless steel, nickel titanium alloys, carbon-filled nylon, carbon-filled polyetheretherketone, polypropylene, high density polyethylene, combinations thereof, or mixtures of one or more of such materials.

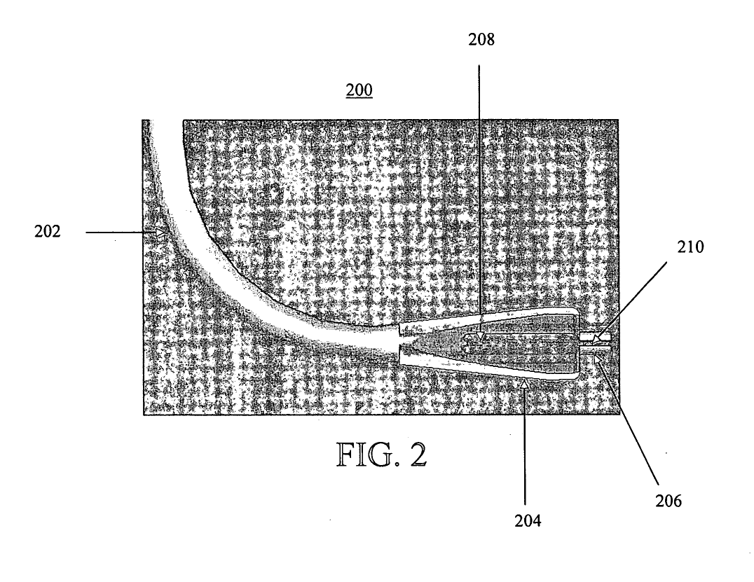

As described briefly above, the device may also be flexible for use in percutaneous procedures, for example, as shown in FIG. 2. In this variation, the device (200) comprises a flexible shaft (202). Any suitable material may be used to construct the shaft. For example, the flexible shaft may be made from nylon, nylon blends, nickel titanium alloys, polyethlyene, polyetheretherketone, polyether block amides, polytetrafluoroethylene, fluorinated ethylene propylene copolymer, stainless steels, polymer blends with or without a supporting metal braid or coil, combinations thereof, and mixtures of one or more of such materials.

In the variation shown in FIG. 2, details of the distal end of the shaft can be seen. For example, the device further comprises a distal tip shoulder (204). The distal tip shoulder (204) has a larger profile than the distal tip (206) of the shaft, which extends distally beyond the distal tip shoulder (204). Having the distal tip (206) of the shaft extend distally beyond distal tip shoulder (204) may be quite useful, because it provides a slight indentation or mild penetration of the device into the tissue and allows the anchor to deploy into the tissue with greater apposition. Similarly, having a distal tip shoulder (204) with a larger profile than the distal tip of the shaft may be quite useful in preventing excessive tissue penetration or excessive tissue damage by the distal tip of the shaft, because such a tip shoulder has a larger footprint of contact with the tissue, thereby distributing the appositional force over a larger surface area. In some variations, the portion of the shaft extending beyond the distal tip shoulder (204) has a smaller outer diameter than the portion of the shaft not extending beyond the distal tip shoulder (204). The distal tip of the shaft may extend beyond the distal tip shoulder by any desirable distance, and this spacing or length generally depends upon the anatomy of the structure receiving the anchor. For example, in variations where the anchors are used to repair the mitral valve, the distal tip may extend beyond the distal tip shoulder by about 0.5 mm to about 2 mm, or about 1 mm to about 3 mm.

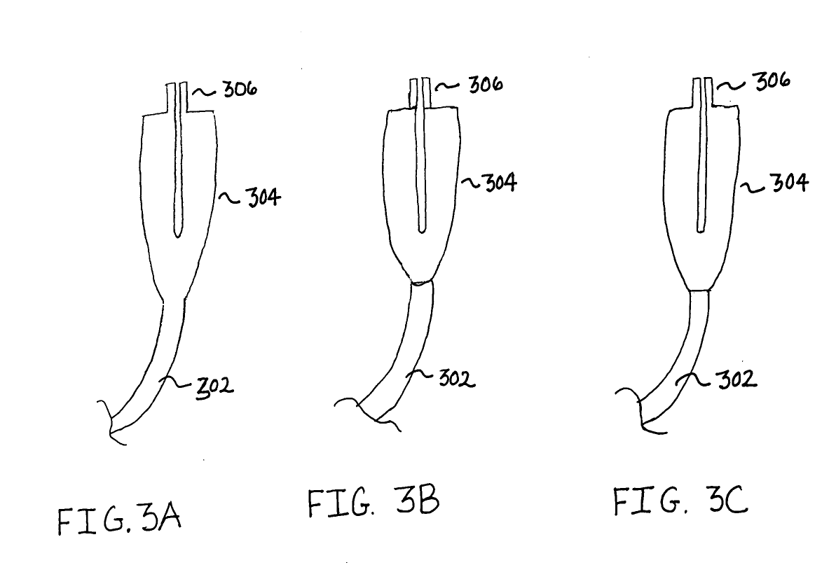

It should be noted, that the distal tip shoulder (204) and distal tip (206) of the shaft may be a single integral unit of shaft (202), although they need not be. For example, distal tip shoulder (204) and distal tip (206) of the shaft may be separate from shaft (202), and connected to the shaft by soldering, welding, gluing (or using another adhesive such as cement), snap-fitting, or by any other suitable mechanism. Similarly, one or the other (e.g., the distal tip shoulder or the distal tip) may be integral with the shaft while the other is not. These variations are depicted in FIGS. 3A-3C. FIG. 3A, for example shows a variation where the distal tip shoulder (304), distal tip (306), and shaft (302) are all a single integral unit. FIG. 3B shows a variation where the distal tip shoulder (304), distal tip (306), and shaft (302) are each separate and distinct structures, and FIG. 3C shows a variation where the distal tip shoulder (304) and distal tip (306) are integral, but separate and distinct from shaft (302).

Referring back to FIG. 2, the distal tip (206) may have a slot (210) therethrough, as may the distal tip shoulder (204). Slot (208) and slot (210) may also be axially aligned. In this way, an anchor may be loaded through the slot (210) in the distal tip (206) and compressed into the slot (208) in the distal tip shoulder (204). The slots (208, 210) may also be useful in allowing a linking member (e.g., a suture, a piece of nylon, a thread or tether, a piece of DACRON® polyester, etc.) to pass through the slots and through the eyelet of the anchor, as will be described in more detail below.

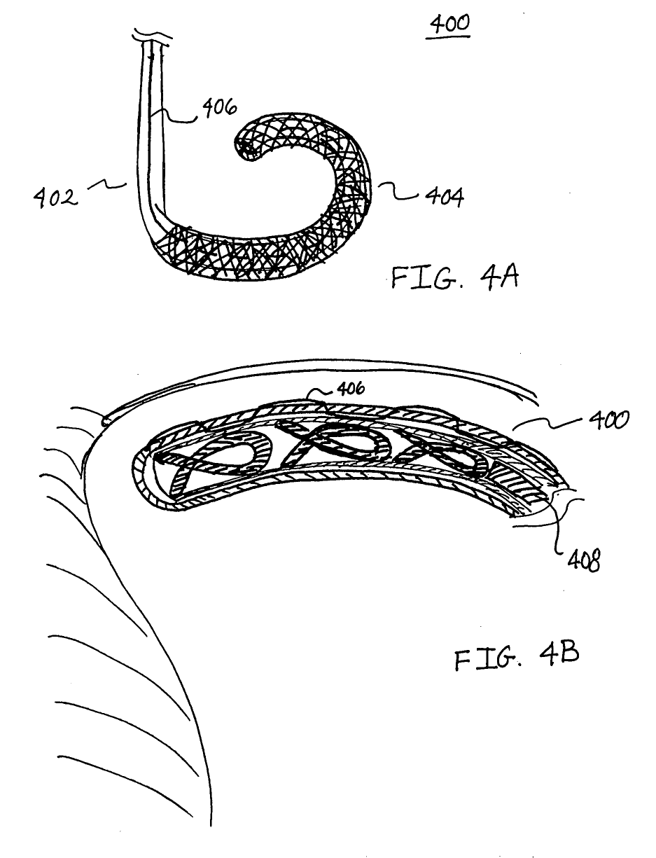

The device may also have a sleeve surrounding (or releasably coupled to) at least a portion of shaft (202). This is especially useful in the percutaneous variations, where the shaft is made of a flexible material. The sleeve may be made from a braided polyester, a woven polyester, polyurethane, polypropylene, a nickel titanium alloy, stainless steel, expanded polytetrafluroethylene, or mixtures thereof. The sleeve may also be of any appropriate length or size. This variation is shown in FIGS. 4A and 4B.

Shown in FIGS. 4A and 4B is a flexible delivery device (400) intended for use in percutaneous procedures. Shaft (402) is surrounded at its distal end by sleeve (404). The length of the sleeve (404) typically corresponds to length of the anchors disposed within the lumen of shaft (402). That is, the sleeve typically covers only that portion of the shaft having anchors housed therein. As described in more detail below, this is because the anchors are deployed through the sleeve and connect the sleeve to the target tissue, or anchor deployment, site.

The sleeve may be particularly useful in promoting tissue ingrowth and in adding extra integrity and structure to the target tissue site. Thus, if there are more anchors than there is length of sleeve, there will be at least a portion of the target tissue site that will not be covered or secured by anchors with the sleeve. Similarly, if there is more length of sleeve than there are anchors, there will be residual slack remaining in the sleeve. This however, may be cured by cutting the sleeve short, as will be described in more detail below.

Also shown in FIGS. 4A and 4B is a cinching strand, or tether (406). This cinching strand may be made of any suitable material. For example, the cinching strand may be a suture, thread, tether, string, and the like, made from materials such as a nickel titanium alloy, stainless steel, polypropylene, polyethylene, polytetrafluoroethylene, nylon, a KEVLAR® brand fiber, a VECTRAN® brand fiber, and the like. The cinching strand is connected to the sleeve (404) and is used to tighten the slack between the anchors after they are deployed, for example, in order to reduce the circumference of a valve annulus, hollow body organ, or the like. FIG. 4B, a cutaway of device (400), also depicts a mechanism for deploying an anchor distally from the lumen of the shaft, shown here as plunger (408).

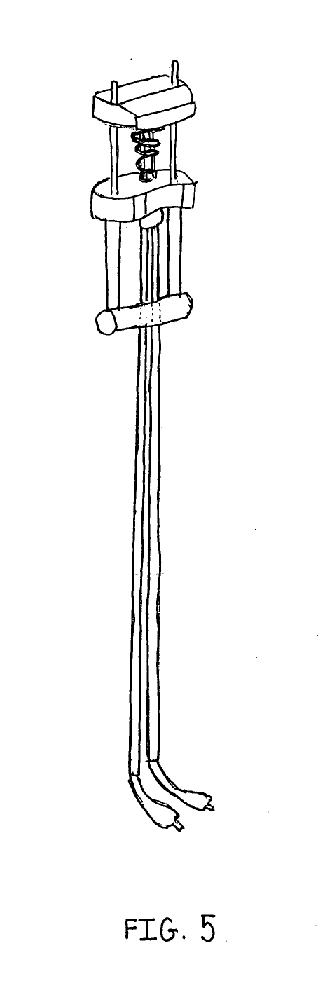

Both the surgical (rigid) and percutaneous (flexible) devices described here may comprise more than one shaft, for example, as shown in FIG. 5 (shown there having two shafts). In this way multiple anchors may be deployed simultaneously. Similarly, multiple anchors may be pre-loaded into a single shaft (e.g., as shown in FIGS. 4B, 6, and 7), and deployed from the shaft, serially or sequentially. In a like manner, the device may also comprise an additional shaft, or an additional lumen within a single shaft, which is configured to inflate a balloon. This for example, may aid in the deployment of the tissue anchors as the inflation of the balloon presses the anchors against the tissue to provide greater apposition. The balloon may be made from a material selected from the group consisting of nylon, polyethylene, polyurethane, combinations thereof, and mixtures of one or more of such materials.

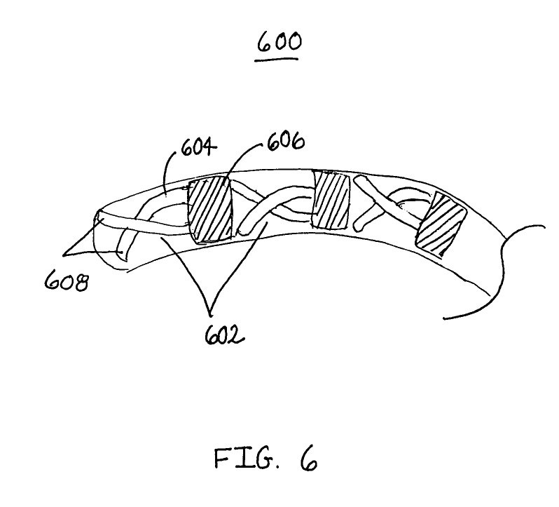

In variations where the anchors are deployed from a shaft serially, it may be useful for the anchors to be potted in a polymer. For example, the anchors (602) shown in FIG. 6 are pre-loaded into the lumen of the shaft of a delivery device (600) for serially deployment. In this figure, at least a portion of the eyelet (604) has been dipped, coated, or potted with a polymer (606) or other suitable material. In this way, the legs or tips of the anchors (608) have an abutment surface, useful in the loading of multiple anchors. Any suitable biocompatible polymer or material may be used to produce this abutment surface. For example, suitable materials include nylon, nylon blends, nickel titanium alloys, polyethlyene, polyetheretherketone, polyether block amides, polytetrafluoroethylene, and the like.

Methods

Methods for deploying a tissue anchor are also described here. As described briefly above, these methods may be used in a variety of surgical or percutaneous procedures. For example, these methods may be quite useful in delivering anchors to a site to reduce the circumference of a hollow body organ, to reduce the circumference of a valve annulus, to close wounds, and the like.

In some variations, the method comprises loading an anchor within a lumen of a shaft (where the anchor comprises an eyelet and the shaft has a slot therethrough), passing a linking member through the slot and through the eyelet of the anchor, and deploying the anchor. The shaft may be rigid or flexible as described above. The linking member may be a suture, tether, thread, string, piece of fabric, or any other material suitable for linking one anchor to another anchor. It should be noted that the loading of the anchor may be performed at any point prior to the delivery. Thus, for example, the devices may come with one or more pre-loaded anchors therein, ready for deployment.

The manner in which the anchor is loaded into the device, depends on the particular configuration of the anchor used. For example, when the anchors to be loaded are those anchors depicted in FIG. 4B, and described in commonly owned co-pending application entitled, “Devices and Methods for Anchoring Tissue” filed on Aug. ______, 2005 (mentioned above), the anchor is typically back loaded into the device. That is, the anchors are aligned and pulled or pushed into the tip of the device, with the anchor legs aligned parallel to the shaft so that the tips of the anchor legs are flush with the tip of the device. A loading tool may be useful in this respect (such as a wire, or other device useful in loading the anchor). After the anchor is loaded into the tip of the device, the linking member is passed through the slot on the distal tip shoulder, and through the eyelet of the anchor. The device is then positioned adjacent to the target tissue or anchor deployment site, and the anchors are deployed by use of the anchor deployment mechanism (e.g., by depressing a handle that actuates a plunger, by pushing on a cable, etc.). The linking member is then removed from the slot of the device, the device is withdrawn from the target site, and the anchor may then be checked for proper placement. After ensuring proper placement of the anchors, these steps are repeated as necessary.

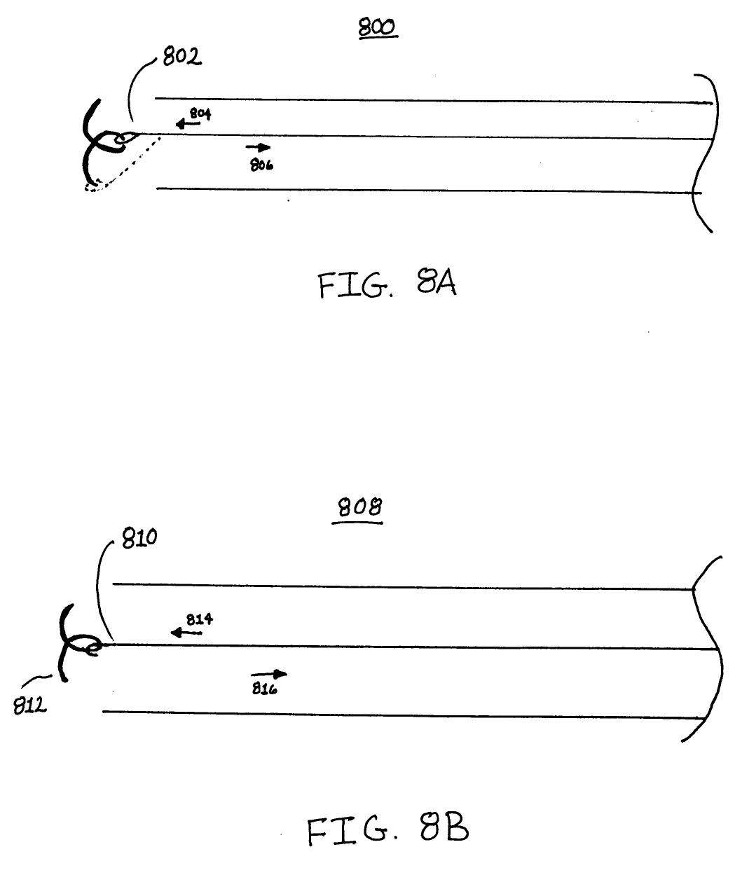

If the anchor was not properly placed, however, it may be retrieved using the anchor deployment device. That is, the anchor may be compressed back down to its collapsed configuration and drawn back into the lumen of the device shaft. Any number of suitable devices or component parts may be useful in the retrieval process. For example, as shown in FIG. 8A, in some variations the anchor is loaded into the device (800) after first being coupled to a looped string or suture (802). In this variation, the looped string (802) is pulled distally (804) out of device (800), threaded onto one leg of the anchor (shown in FIG. 8A by dashed lines), and then slid around the anchor until it reaches, or is positioned about, the eyelet. Once the looped string has been properly threaded, the anchor is loaded into the device by pulling proximally (806) on looped string (802). Here, if the anchor has not been properly deployed or placed, proximal pulling on the looped string will cause the legs to collapse against the deployment device (800), and allow the anchor to be pulled therein. The looped string may also function to help with proper alignment and loading of the anchor into the device.

In another variation, shown in FIG. 8B, the deployment device (808) further comprises a pull-push wire (810). In a manner similar to that described with respect to FIG. 8A above, the anchor (812) is first loaded or threaded onto push-pull wire (810). This is accomplished, by pushing push-pull wire (810) distally (814) out of device (808), and then loading anchor (812) onto push-pull wire (810) such that the distal hook of the push-pull wire (810) is threaded through the eyelet of the anchor (812). The anchor is then loaded into the device (808) by proximal pulling (816) of push-pull wire (810). In a manner similar to that described above with respect to FIG. 8A, if the anchor (812) is misdeployed or improperly placed, it can be withdrawn back into delivery device (808) by proximal pulling on push-pull wire (810). As with the variation described above, the push-pull wire may also function to help with proper alignment and loading of the anchor into the device.

To help with proper placement, the linking member may be marked. For example, in some procedures, it may be desirable to have the anchors spaced apart evenly (e.g., 1 mm to 5 mm apart). Instead of requiring the guesswork of the operator, the linking member may be marked periodically to indicate where the next anchor should be deployed.

Other methods of deploying a tissue anchor comprise loading an anchor within a lumen of a shaft (where the anchor comprises an eyelet), and deploying the anchor distally from the lumen. In this variation, the inner diameter of the lumen is smaller than the diameter of the eyelet of the anchor to be disposed therein when the anchor is in an expanded configuration. As described briefly above, in some variations, this allows the legs of the anchor to assume a more linear shape as the legs are first deployed from the tip of the lumen, the legs of the anchor curving upon expansion and full deployment.

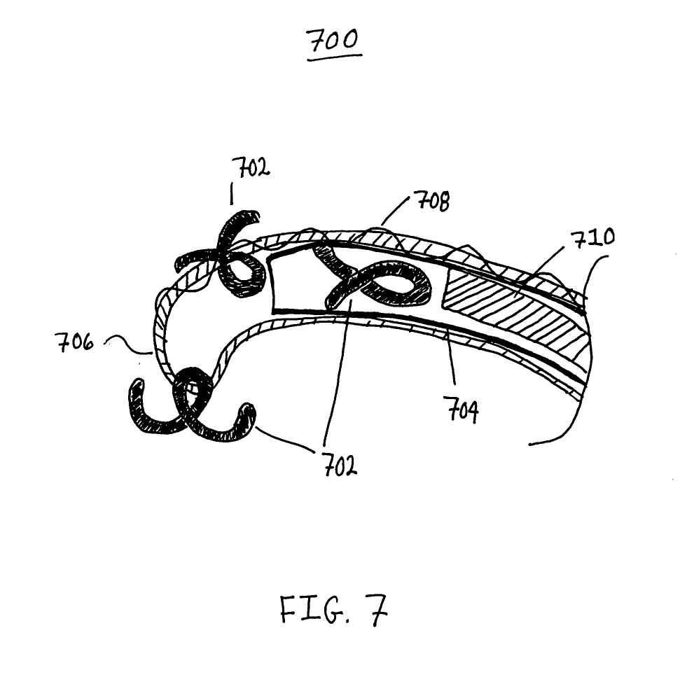

FIG. 7 provides a depiction of this variation, where a percutaneous device is used to deliver anchors from the distal end of a shaft. Shown in FIG. 7 is device (700) having anchors (702) disposed within shaft (704), sleeve (706), cinching member or tether (708), and push cable (710). As shown, the anchors are back loaded into the lumen of shaft (704), and as the push cable is moved distally forward, the anchors are deployed from the distal end of the shaft. In this variation, there is no linking member. As an anchor is deployed from the distal end of the shaft, it pierces through and catches the sleeve, pulling it to the target tissue or anchor deployment site. The shaft is then repositioned to the next desirable target tissue site location, and the process is repeated. In this way, the anchors secure the sleeve to the tissue. After all the anchors have been deployed, cinching cable (708) is pulled proximally, to cinch the anchors and sleeve in a purse string fashion. The cinching cable is then cut and secured. In the event there is excess sleeve, the sleeve may also be cut. The device is then withdrawn.

When the flexible percutaneous devices described here are used in the repair of a heart valve (e.g., a mitral valve), and particularly the repair of a valve where the valve is approached subannularly, the catheter shaft typically has a radius of curvature that is larger than that of the annulus of the valve. In this way, when the catheter is situated in the subannular groove (i.e., the circular track defined by the joinder of the horizontal underside of the valve annulus with the ventricle wall,), the tip of the catheter will point outward against the annulus and the ventricular wall. The tip of the catheter may also be beveled to help direct the anchors outward.

Kits

Kits for the deployment of tissue anchors are also described here. In some variations, the kits comprise a device comprising a shaft configured to deploy anchors distally therefrom, and a linking material configured to couple the anchors together. As set forth above, the linking material may be any suitable material configured to couple the anchors together.

In some variations the linking material is a suture made of any suitable material, such as those described above with respect to the cinching strand. The suture may include one marking, or a series of markings to help indicate the proper placement or spacing of the anchors as mentioned above. For example, the suture may be marked every few centimeters to make it easy for the user to know how far apart to space the next anchor.

The device of the kit may be any of those devices described above. The kits may also include instructions on how to use the contents of the kit. The components of the kit may be packaged together or separately.

What is claimed is:

1. A device for the deployment of tissue anchors comprising:
a shaft defining a lumen for housing at least one anchor therein, the anchor having an eyelet; and
a mechanism for deploying the anchor distally from the lumen, wherein the inner diameter of the lumen is the same size or smaller than the diameter of the eyelet of the anchor to be disposed therein when the anchor is in an expanded configuration.

2. The device of claim 1 wherein the distal tip of the shaft has a slot therethrough.

3. The device of claim 2 further comprising a distal tip shoulder.

4. The device of claim 3 wherein the distal tip shoulder has a slot therethrough.

5. The device of claim 4 wherein the distal tip shoulder slot and the distal tip of the shaft slot are axially aligned.

6. The device of claim 1 further comprising a distal tip shoulder, wherein the distal tip shoulder comprises a region of the shaft.

7. The device of claim 3 wherein the distal tip of the shaft extends beyond the distal tip shoulder.

8. The device of claim 7 wherein the portion of the shaft extending beyond the distal tip shoulder has a smaller outer diameter than the portion of the shaft not extending beyond the distal tip shoulder.

9. The device of claim 7 wherein the distal tip of the shaft extends beyond the distal tip shoulder by about 1 to about 3 mm.

10. The device of claim 1 wherein the shaft is made of a flexible material.

11. The device of claim 10, wherein the material is selected from the group consisting of nylon, nylon blends, nickel titanium alloys, polyethlyene, polyetheretherketone, polyether block amides, polytetrafluoroethylene, fluorinated ethylene propylene copolymer, stainless steels, polymer blends with or without a supporting metal braid or coil, combinations thereof, and mixtures of one or more of such materials.

12. The device of claim 10 wherein a sleeve surrounds at least a portion of the shaft.

13. The device of claim 12 wherein the sleeve is made from a material selected from the group consisting of a braided polyester, a woven polyester, polyurethane, polypropylene, a nickel titanium alloy, stainless steel, expanded polytetrafluoroethylene, combinations thereof, and mixtures of one or more of such materials.

14. The device of claim 10 further comprising an additional lumen configured for inflation of a balloon near its distal end.

15. The device of claim 13 where the balloon is made of a material selected from the group consisting of nylon, polyethylene, polyurethane, combinations thereof, and mixtures of one or more of such materials.

16. The device of claim 1 wherein the shaft is made of a rigid material.

17. The device of claim 16, wherein the rigid material is selected from the group consisting of stainless steel, nickel titanium alloys, carbon-filled nylon, carbon-filled polyetheretherketone, polypropylene, high density polyethylene, combinations thereof, and mixtures of one or more of such materials.

18. The device of claim 16 wherein the shaft has a curve near its distal tip.

19. The device of claim 18 wherein the curve has an angle ranging from about 15 to about 90 degrees.

20. The device of claim 1 comprising at least two shafts.

21. The device of claim 1 wherein the shaft is configured to receive at least two anchors therein.

22. The device of claim 1 wherein the mechanism is a plunger slidably disposed within at least a portion of the lumen, the device further comprising an actuator for actuating the plunger.

23. A kit for the deployment of tissue anchors comprising:
a device comprising a shaft configured to deploy anchors distally therefrom; and
a linking material configured to couple the anchors together.

24. The kit of claim 23 further comprising instructions on using the kit.

25. The kit of claim 23 wherein the linking material is a suture.

26. The kit of claim 23 wherein the device is the device of claim 1.

27. A method for deploying a tissue anchor comprising:
loading an anchor within a lumen of a shaft, wherein the anchor comprises an eyelet, and the shaft has a slot therethrough;
passing a linking member through the slot and through the eyelet of the anchor; and
deploying the anchor.

28. The method of claim 27 further comprising placing a sleeve over the linking member.

29. The method of claim 28 wherein the step of placing a sleeve comprises sliding a sleeve over the linking member.

30. The method of claim 27 further comprising retrieving the anchor in the event of misplacement.

31. A method for deploying a tissue anchor comprising:
loading an anchor within a lumen of a shaft, wherein the anchor comprises an eyelet; and
deploying the anchor distally from the lumen, wherein the inner diameter of the lumen is the same size or smaller than the diameter of the eyelet of the anchor to be disposed therein when the anchor is in an expanded configuration.

32. The method of claim 31 wherein deploying the anchor distally from the lumen further comprises deploying the anchor distally from the lumen into a sleeve and the tissue, wherein the sleeve is releasably coupled to the shaft and wherein the anchor couples the sleeve to the tissue.

33. The method of claim 32 wherein the sleeve comprises a cinching member, and further comprising applying tension to the cinching member.

34. A method for deploying a tissue anchor comprising:
loading an anchor within a lumen of a shaft; and
deploying the anchor distally from the lumen, wherein deploying the anchor distally from the lumen further comprises deploying the anchor distally from the lumen into a sleeve and the tissue, wherein the sleeve is releasably coupled to the shaft and wherein the anchor couples the sleeve to the tissue.
